(19) United States
(12) Patent Application Publication
(10) Pub. No.: US 2017/0203119 A1
(43) Pub. Date: Jul. 20, 2017

(54) PHOTOELECTRIC RESONANCE CHIP AND METHOD FOR CONDUCTING MICROWAVES THROUGH THE SAME

(71) Applicants: JIN-SHENG TSENG, NEW TAIPEI CITY (TW); JING-WEN LIAO, NEW TAIPEI CITY (TW); SHU-HUA SHIAO, NEW TAIPEI CITY (TW)

(72) Inventors: JIN-SHENG TSENG, NEW TAIPEI CITY (TW); JING-WEN LIAO, NEW TAIPEI CITY (TW); SHU-HUA SHIAO, NEW TAIPEI CITY (TW)

(21) Appl. No.: 15/157,413

(22) Filed: May 18, 2016

(30) Foreign Application Priority Data
Jan. 15, 2016 (TW) 105200621

(51) Int. Cl. A61N 5/04; A61N 5/06; A61N 1/36

(52) U.S. Cl. CPC A61N 5/04; A61N 1/36014; A61N 5/0613; A61N 2005/0645

(57) ABSTRACT

A photoelectric resonance chip includes a chip main body further including an absorption portion, strengthening device, emission portion, and a light wave generating element and electric wave generating element are configured on one side of the chip main body. When a user carry the chip on their body, the strengthening device enlarge the power of external microwaves absorbed by the absorption portion, and the emission portion further feeds the microwaves with the enlarged power back to the human body. Thereafter, the fed-back microwaves carry the light wave energy and electric wave energy to flow around the whole body to strengthen human body's bioelectrical and the effect of the light wave energy and electric wave energy to the human body.

### TECHNICAL FIELD OF THE INVENTION

The present invention relates to a photoelectric resonance chip and a method for conducting microwaves through the same, using microwave conduction to achieve the object of feeding a local action back to a whole human body, while possessing the effects of light wave energy and electric wave energy.

### DESCRIPTION OF THE PRIOR ART

Health patches are patches attached to a part of human body wanting to be improved and emitting far infrared through magnets therein to strengthen the circulation of a user's body, thereby improving the body's condition.

Such kinds of health patches have some problems in use:
1. magnets themselves are radioactive, being possibly dangerous to cause damage to a human body, and the radioactivity thereof will decay gradually in a long term use, and finally, will disappear completely to lose the effect.
2. the energy cannot be concentrated effectively because the wavelength bandwidths of the magnets themselves are too broad such that the effect of improving human health is limited; although large machines such as far infrared generators whose bandwidths are rather concentrated are available in the market, they are not portable and incapable of being in prolonged contact with a human body to make far infrared effective such that they do not conform to modern people's requirements.
3. because the effective ranges of a conventional health patch is rather small and it can only effect locally in the vicinity thereof, more patches are required to attach one by one to a human body if many places on the body or the whole body need be conditioned.

### SUMMARY OF THE INVENTION

To overcome the defects of conventional health patches mentioned above, for example, lower repeated usage, necessary direct contact with a human body, rather local effect, and more complicated operation, the present invention is proposed.

One object of the present invention is to provide a photoelectric resonance chip and a method for conducting microwaves through the chip, utilizing actions such as absorption, strengthening and feeding-back to conduct microwaves to a human body, and adding light wave energy and electric wave energy in the microwaves to increase human body's bioelectrical energy, and at same time to apply the light wave energy and electric wave energy to the whole body with the microwaves.

To achieve the object mentioned above, the present invention propose a photoelectric resonance chip and a method for conducting microwaves through the chip, where the photoelectric resonance chip includes a chip main body further including an absorption portion, strengthening device, emission portion, and a light wave generating element and electric wave generating element are configured on one side of the chip main body. When a user carries the chip on their body, the strengthening device enlarge the power of external microwaves absorbed by the absorption portion, and the emission portion further feeds the microwaves with the enlarged power back to the human body. Thereafter, the fed-back microwaves carry the light wave energy and electric wave energy to flow around the whole body to strengthen human body's bioelectrical and the effect of the light wave energy and electric wave energy to the human body.

### BRIEF DESCRIPTION OF THE DRAWINGS

FIG. 1 is a perspective view of the present invention;

FIG. 2 is a perspective view of the present invention, showing components configured inside a chip main body which is drawn with a dotted line;

FIG. 3 is a flow chart of the present invention;

FIGS. 4 and 5 respectively are a schematic view of the present invention upon implementation; and

FIG. 6 is a schematic view of the present invention configured with a fixing element upon implementation.

### DETAILED DESCRIPTION OF THE PREFERRED EMBODIMENTS

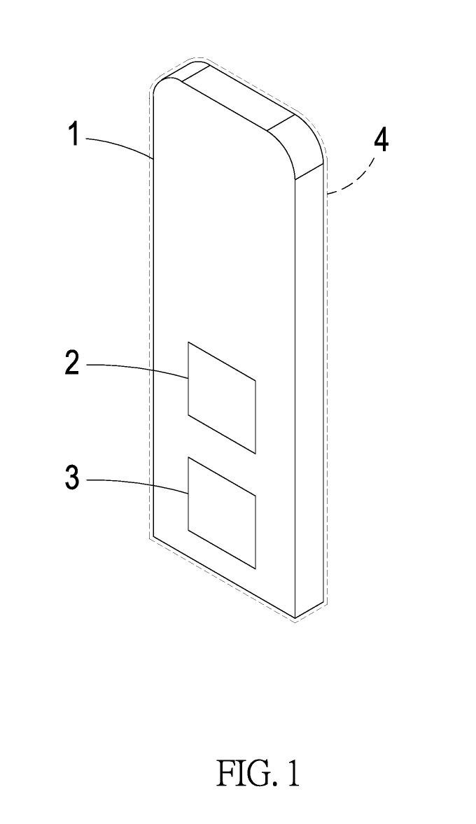

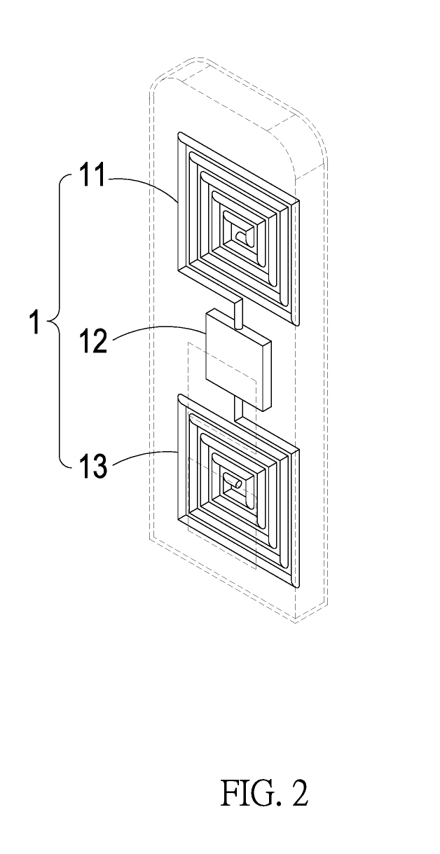

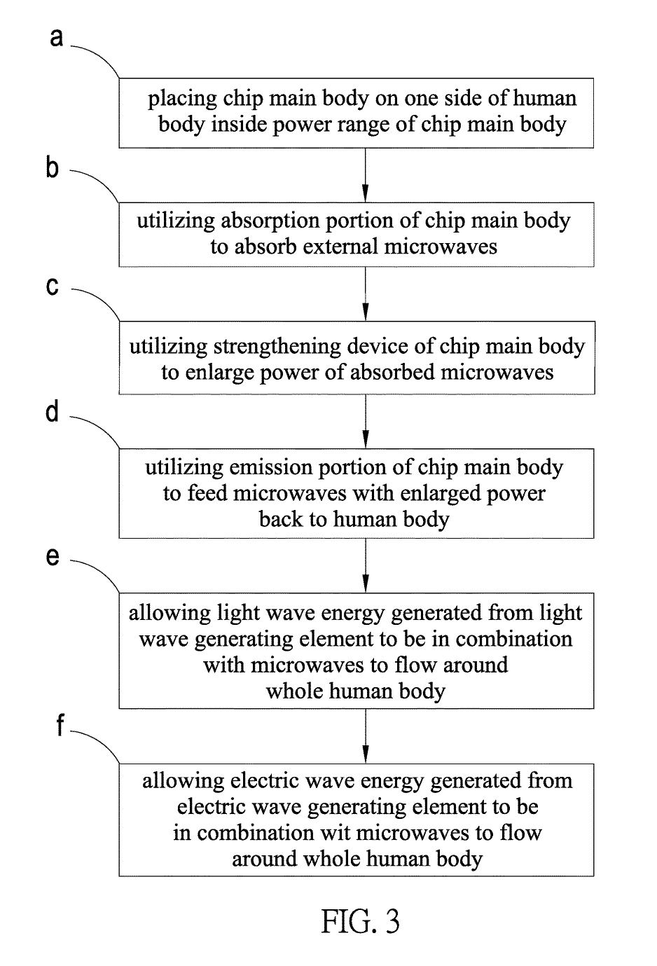

Referring to FIGS. 1 to 3, a photoelectric resonance chip according to the present invention includes a chip main body 1, at least one light wave generating element 2, at least one electric wave generating element 3 and at least one protective shield 4.

The chip main body 1 includes an absorption portion 11 for the absorption of external micro waves, a strengthening device 12 in connection with the absorption device 11 and adapted to enlarge the power of the received microwaves and an emission portion 13 in connection with the strengthening device 12 and adapted to feed the microwaves with the enlarged power back to a human body. Here, the microwaves are ranged from 0.01 milliamperes(mA) to 10 milliamperes(mA).

The light wave generating element 2 is configured on one side of the chip main body 1 and used to generating light wave energy capable of being in combination with the microwaves.

The electric wave element 3 is configured on one side of the chip main body 1, and used to generate electric wave energy capable of being in combination with the microwaves.

The protective shield 4 covers the chip main body 1 and may be made of flexible material, rigid material or nanometer material.

A method for conducting microwaves through a photoelectric resonance chip according to the present invention includes the steps as following:
(a) placing the chip main body on one side of a human body in the range of the power of a chip main body;
(b) using the absorption portion of the chip main body to absorb external microwaves;
(c) enlarging the power of the absorbed micro waves through the strengthening device of the chip main body;
(d) feeding the absorbed microwaves with the enlarged power nsvl to the human body through the emission portion of the chip main body;
(e) allowing light wave energy generated from the light wave generating element to be in combination with the microwaves to flow around the whole human body; and
(f) allowing electric wave energy generated from the electric wave generating element to be in combination with the microwaves to flow around the whole human body.

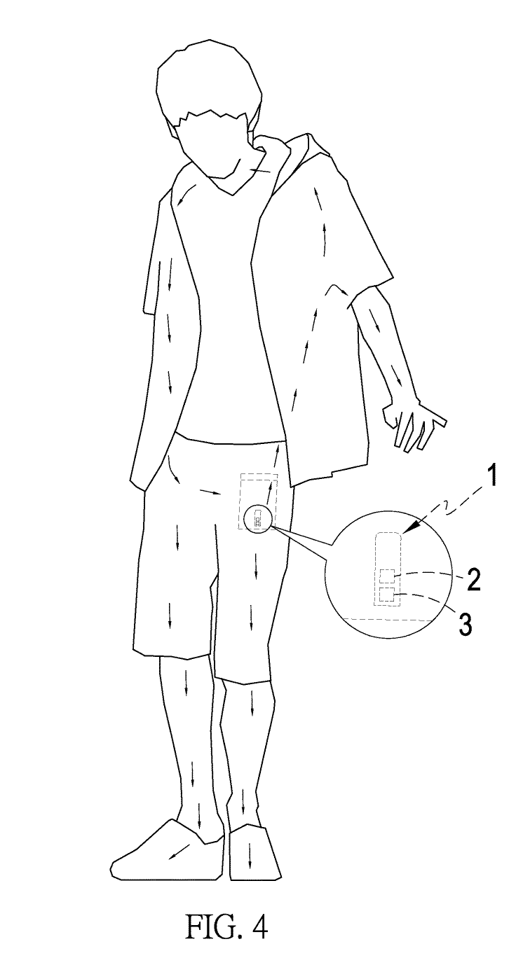

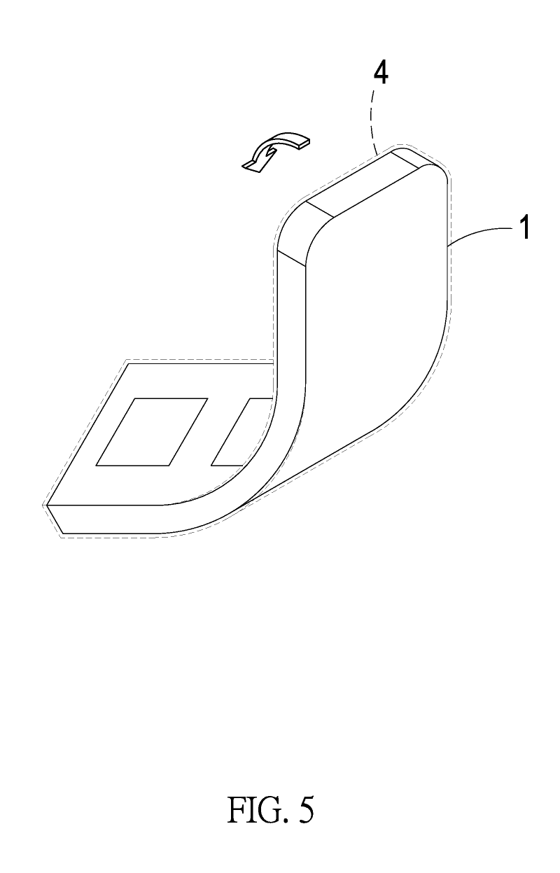

The present invention, as FIGS. 1 to 5 show, is a chip structure which is simply structured, lightweight and easy to carry; the area thereof approximately is smaller than 25 cm². In one preferred embodiment, the chip is a small area body which is 4 cm in length and 2 cm in width suitable for a user to carry and occupying no space. Furthermore, because the microwave radiation energy generated from the chip main body is very low, approximately one-thousandth of a Bluetooth earphone (currently, the safety standard for mobile phones announced by the US Federal Communication Committee is 1.6 w/kg, the safety standard of Europe is 2.0 w/kg recommended by World Health Organization, and the radiation energy of a Bluetooth earphone only is one-thirtieth of the standard value), it has no negative impact to a human body.

In actual use, a user can use the chip of the present invention only by placing it in a pocket or combining it with a wearing object such as clothes, a hat or a necklace, and the chip can then be close to a human body's surface naturally because the length of the chip main body is approximately ranged from 1 cm to 4 cm. In this situation, the absorption portion 11 of the chip main body 1 is used to absorb external microwaves including weak electric waves in the environment and weak electric waves in the human body such that it is unnecessary to attach the chip main body 1 close to the human body and the absorption action can then be carried out. Thereafter, the microwaves are conducted to the strengthening device 12 and the enlargement of the power of the microwaves is carried out therein. Next, the microwaves with the enlarged power are fed back to the human body through the emission portion 3. Furthermore, the microwaves are allowed to carry the light wave energy and electric wave energy generated respectively by the light wave generating element 2 and electric wave generating element 3 by means of co-frequency resonance in such a way to allow the microwaves themselves together with the light wave energy and electric wave energy acted locally to flow around the whole human body. Furthermore, the microwaves can generate toroidal magnetic fields which can speed blood flow; the formation of the toroidal magnetic fields is similar to adding many pipeline booster pumps in a blood system; the toroidal magnetic fields so generated have not only a thrusting action to speed blood but a moving action to move all cells in main and collateral channels such that they can strengthen bioelectrical and speed cell repair. As a result, a user can improve the circulation condition adjacent to the chip and achieve the effects of improving the human body's health, for example, strengthening the human body's main and collateral channels and acupuncture points, improving blood flow speed and volume inside blood vessels through the microwaves emitted from the user themselves and the cyclic operation mentioned above. When the health condition of a human body is improved, the microwaves emitted therefrom will be strengthened gradually, and the circulation will be more strengthened by means of the method mentioned above in such a way to increase local temperature of the human body, and further to achieve the efficiencies of activating internal organs, enhancing immunity, increasing basal metabolic rate, improving blood circulation and adjusting the autonomic nerves, the function of hormonal balance and other effects. Thereupon, the human body health condition will further be improved with diet control and moderate exercise.

Furthermore, since the volume of the chip of the present invention is small, if the point of a human body where it is placed is close to a specific acupuncture point, not only a systemic effect can be generated through the microwaves flowing around the whole body, but the efficiency of acupuncture point stimulation can be brought into full play. The Acupuncture points are sensitive and functional points on main and collateral channels and contacts of bioelectrical, and main and collateral channels are the channels for the transmission of bioelectrical. An electric voltage and potential will be changed when a magnetic field is applied on an acupuncture point to excite bioelectrical to generate electromagnetic waves, and the generated electromagnetic waves are transmitted to the main and collateral channels of the whole body to form stimulation to the central nerve when they are transmitted thereto, thereby adjusting a lesion or lesions.

The co-frequency resonance principle mentioned above is utilizing the special physical function of co-frequency oscillation, allowing light particles to impact the molecules of the molecular structure of all organisms at a light speed without utilizing any media, strengthening the molecular structure to generate a vibration action immediately, and achieving the activation of the molecules. If it is explained with quantum mechanics, the phenomenon mentioned above is called photoelectric effect. According to the known basic scientific theories, all organisms (including human body) are constituted by molecules formed by the gathering of atoms and having their own specific vibration frequencies. Thereupon, the vibration frequency of a human body is interacted synchronously with co-frequency resonance energy, allowing the energy of the body to be increased. In addition, the chip body 1 absorbing external microwaves and electromagnetic waves can decrease the interference of the electromagnetic field generated from computer, mobile phone, or the like to a human body. Whereby, the present invention can strengthen a user's physical strength, endurance and resistance to stress, as well as, for example, improve the performance of the mind and focus attention.

Furthermore, high body temperature (36.5° C. to 37° C.) caused by the chip inside a human body facilitates the stronger mobility of enzymes; body temperature increases by 1 degree Celsius, the number of pulses per minute will increase by 10, resulting in the body being full of fresh oxygen, cell metabolism being also increased. In addition, high body temperature may also make blood vessels become softer such that blood can be transmitted to the whole body more smoothly; if the temperature of a human body can be kept uniform, the autonomic nervous and hormonal secretion will also be maintained balanced, and the normal functions thereof can be brought into full play. Thereupon, the present invention allowing local temperature to be increased can facilitate a human body's health.

Because the strengthening device 12 is a passive element, the whole chip receives microwaves emitted from a human body or existing in the environment in a passive way such that there is no radioactive decay and no radioactivity to hurt the human body. In addition, the action way of receiving microwaves passively allows the chip to be used forever without an electricity supply problem. Furthermore, the chip main body 1 is prevented from being exposed directly to and damaged by the atmosphere because the protective shield 4 carries out the protection, thereby allowing the whole chip to be more durable, small-sized. Since the protective shield 4 is made of nanometer material, the chip main body 1 may have certain flexibility such that it is not affect by deformation such as compression or curling while being placed inside a pocket or clothes and can be used normally.

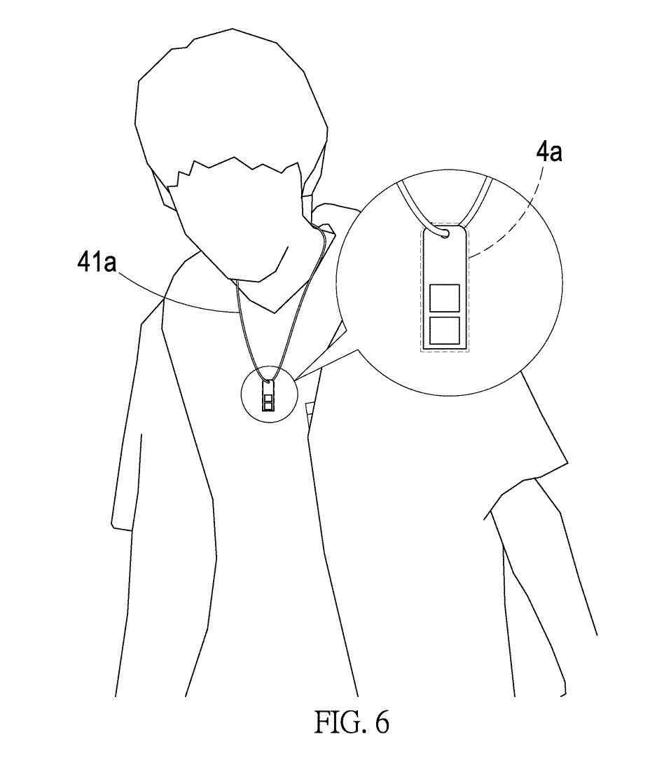

Referring to FIG. 6, the protective shield 4a is made of rigid material, on which a fixing element 41a is configured additionally, where the fixing element 41a is adapted to fix to a human body. Specifically, the fixing element 41a may be a rope or strap which is replaceable depending on use requirements; the fixing element 41a, shown in the figure, is a longer rope, and the protective shield 4a is modeled, allowing more flexible use collocation thereof.

Therefore, the present invention has the following advantages compared with the prior art:
1. the emission portion 13 is used to generate microwaves similar to the ones of a human body, and the microwaves so generated are applied locally to a human body and further allowed to flow around the whole body with the bioelectrical of the body.
2. the microwaves carry the light wave energy and electric wave energy to flow around the whole body and further to condition all parts of the body.
3. the absorption portion 11 is used to absorb the microwaves of a human body and the environment, the strengthening device 12 is used to enlarge the power thereof, and the microwaves with the enlarged power are fed back to the body through the emission portion13 in such a way to strengthening the body's bioelectrical to circulate the whole body.
4. the chip main body 1 is placed on an appointed acupuncture point, and the conditioning of the Meridian and acupuncture point can then be strengthened particularly.
5. the protective shield 4 is used to protect the chip main body 1, allowing the chip main body 1 not to be in contact with the atmosphere, thereby protecting the whole structure, making the structure dependable. Especially, when nanometer material is used, the protective shield 4 will be compression-resistant and flexible.
6. because the absorption portion 11, strengthening device 12 and emission portion 13 are combined inside the chip main body 1 without needing an extra substrate to be used as a carrier, the overall volume can be more compact.
7. the chip of the present invention can be used again and again for a long time and collocated with the fixing element 41a which is fixable to the body, allowing the whole to be made into a decoration, thereby making it portable and increasing the collocation flexibility.
8. the present invention can further improve a human body's health by means of the principle of allowing temperature to be increased locally.

I claim:

1. A photoelectric resonance chip, comprising:
a chip main body, comprising an absorption portion for the absorption of external microwaves, a strengthening device in connection with said absorption portion and adapted to enlarge a power of said received microwaves, and an emission portion in connection with said strengthening device and adapted to feed said microwaves with said enlarged power back to said human body;
at least one light wave generating element, configured on one side of said chip main body and adapted to generate light wave energy capable of being in combination with said microwaves; and
at least one electric wave generating element, configured on one side of said chip main body and adapted to generate electric wave energy capable of being in combination with said microwaves.

2. The chip according to claim 1, further comprising at least one protective shield covering said chip main body, and said protective shield is made of flexible material, rigid material or nanometer material.

3. The chip according claim 2, wherein said protective shield has at least one fixing element for the fixation to a human body.

4. The chip according to claim 1, wherein said chip main body is flexible.

5. The chip according to claim 1, wherein said microwaves are ranged from 0.01 millamperes (mA) to 10 milliamperes (mA).

6. A method for conducting microwaves through a photoelectric resonance chip, comprising the following steps:
(a) placing a chip main body on one side of a human body inside a power range of said chip main body;
(b) utilizing an absorption portion of said chip main body to absorb external microwaves;
(c) utilizing a strengthening device of said chip main body to enlarge power of said absorbed microwaves;
(d) utilizing an emission portion of said chip main body to feed said microwaves with said enlarged power back to said human body;
(e) allowing light wave energy generated from a light wave generating element to be in combination with said microwaves to flow around said whole human body; and
(f) allowing electric wave energy generated from an electric wave generating element to be in combination wit said microwaves to flow around said whole human body.

7. The method according to claim 6, wherein said photoelectric resonance chip further comprises at least one protective shield covering said chip main body, and said protective shield is made of flexible material, rigid material or nanometer material.

8. The method according to claim 7, wherein said protective shield has at least one fixing element for the fixation to said human body.

9. The method according to claim 6, wherein said chip main body is flexible.

10. The method according to claim 6, wherein said microwaves are ranged from 0.01 millamperes (mA) to 10 milliamperes (mA).
